(19) United States
(12) Patent Application Publication
(10) Pub. No.: US 2010/0174380 A1
(43) Pub. Date: Jul. 8, 2010

(54) EXTENDED RANGE OF MOTION, CONSTRAINED PROSTHETIC HIP-JOINT

(76) Inventor: Ralph H. Lewis, El Dorado Hills, CA (US)

Correspondence Address: Donald E Schreiber;Donald E Scheiber A Professional Corporation, P O Box 2926, Kings Beach, CA, 96143-2926, US

(21) Appl. No.: 12/312,188

(22) PCT Filed: Nov. 13, 2007
(86) PCT No.: PCT/US07/23838

§ 371 Date: May 8, 2009

Related U.S. Application Data
(60) Provisional application No. 60/858,779, filed on Nov. 13, 2006.

(51) Int. Cl. A61F 2/32

(52) U.S. Cl. 623/22.11

(57) ABSTRACT

A prosthetic hip-joint includes: a. a prosthetic acetabulum cup for implantation into a pelvis; b. a prosthetic femoral assembly which includes: i. a ball-shaped femoral head that during implantation becomes located within the cup; and ii. a femoral stem that is fixed at a first end to the head, and that has a second end distal from the first end which is adapted for implant into a medullary canal of a femur, and c. a liner assembly adapted to he secured to the cup and is also adapted to receive and to constrain the head against dislocation. In one aspect the hip-joint permits the head to rotate through an angle which exceeds at least 153 degrees while concurrently constraining the head against dislocation. In another aspect the hip joint constrains the head a dislocation by a preestablished amount of force which is adjustable during implantation thereof.

### TECHNICAL FIELD

The present invention relates to implantable prosthetic devices for hip-joint replacement in a human body. More particularly, the present disclosure relates to a prosthetic acetabulum cup and liner assembly as well as a prosthetic ball attached to a prosthetic femur, the acetabulum cup and liner assembly controllably constraining the ball in the acetabulum cup and liner assembly.

### BACKGROUND ART

During recent years the number of people requiring a joint replacement has been increasing. A paper entitled “Developing PEEK Polymer as a Bearing Material for Implants,” John Devine, ©2006 Medical Devices & Diagnostic Industry, reports that each year approximately 1.4 million joint replacement procedures are performed worldwide. Of joint replacement procedures, a paper entitled “Failure Analysis of Composite Femoral Components for Hip Arthroplasty,” Chaodi Li, PhD; et al., Journal of Rehabilitation Research & Development, Vol. 40, No. 2, March/April 2003, pp. 131-146, estimated that at the time of its publication 800,000 total hip replacements were be performing annually worldwide. However, as described below it appears that there still exist obstacles to providing a prosthetic hip replacement that matches the original healthy hip joint. A significant concern in hip replacement surgery is the ease with which the prosthetic hip replacement can dislocate immediately after surgery. The range of motion (“ROM”) provided by a prosthetic hip replacement is another concern especially for younger, more active recipients.

The natural human hip is considered a relatively frictionless ball and socket joint that is enclosed by a soft tissue capsule. A ball-like head of the femur rotates within a socket or acetabulum situated in the pelvis. The soft tissue capsule is comprised of ligaments; the ilio-femoral, ischio-femoral and pubo-femoral ligaments being external to the joint while the ligamentum teres is an internal ligament. A primary function of these ligaments is to retain the femur lightly in the acetabulum, prevent extension of the femur much beyond the straight position, and limit the extent of abduction/adduction and movements of rotation.

The ball-like head of the femur is connected to the thigh bone by a neck which is angularly disposed relative to the femoral axis and relative to the vertical axis of the body. Thus any load applied by the body through the hip and femoral neck to the thigh bone and leg and any impact, such as caused by walking, jumping and the like applied by the leg and thigh bone through the femoral neck and hip to the body, is transmitted angularly through the femoral neck. This angular transmission of the load and forces through the femoral neck results in high stresses and high sheer-loads applied to the femoral neck. These high stresses, when normally applied can cause dislocation of the femoral head from the acetabulum or hip socket, and fracture or breaking of the femoral neck. For ninety-five percent (95%) of normal US adults, dislocating the femoral head from the acetabulum requires an estimated force between one hundred and twenty five (125) to two hundred (200) lbs. In older people the femoral neck often becomes brittle, and in both older and younger people is subject to fracture.

Presently, if the natural hip-joint displays an appropriate anomaly, sufficient damage or diseased state, the natural hip-joint is usually replaced by an implantable prosthetic hip-joint replacement. The prosthetic hip-joint replacement includes a substantially spherically-shaped head that is attached to the femur by a neck and stem which fits into the medullary canal. The prosthetic hip-joint replacement also includes a corresponding artificial socket implanted into the acetabulum, which may be suitably enlarged for the purpose. Conventional prosthetic hip-joint sockets normally embody an acetabulum-type cup and liner assembly having a spherically-shaped cavity which receives and rotatably supports the substantially spherically-shaped head. The acetabulum-type cup is suitably secured in various ways to the acetabulum pocket of the pelvis. In this way, the implantable prosthetic hip-joint replacement establishes a ball and socket-type joint which permits ordinary-type of articulated motion provided by the natural hip-joint.

During a hip-joint reconstructive procedure, the ligamentous capsule around the natural hip-joint is usually resected. When the ligaments of the natural joint are resected during the reconstructive procedure, the artificial joint is inherently less stable and subject to dislocation. Consequently, there exists an increased potential for artificial joint dislocation when a total hip-joint prothesis implantation into a patient causes ligamentous laxity. For example, after undergoing a hip replacement procedure a patient is strongly advised to avoid any pressure while in a crossed leg position. Until the ligamentous capsule around the natural hip-joint heals sufficiently, pressure applied in a crossed leg position can easily dislocate the substantially spherically-shaped head from the prosthetic acetabulum cup liner.

In an attempt to prevent dislocation, some total joint implant devices have been designed to constrain the substantially spherically-shaped head within the artificial acetabulum. However, these constrained devices transfer greater forces to the acetabulum cup due to a lever effect that would normally cause dislocation. Therefore, higher stresses occur at the bone/acetabular component interface that results in loosening of the acetabulum cup. U.S. Pat. No. 4,676,798 exemplifies one type of a constrained implantable prosthetic hip-joint.

To replace the natural socket, some prosthetic acetabulum cup assemblies include a metallic shell for attachment to a suitably enlarged acetabulum. Such prosthetic acetabulum cup assemblies may include a polymer bearing which is inserted into the metallic shell that provides a hemispherical bearing surface for receiving the prosthesis’ substantially spherically-shaped head. Frequently, the polymer bearing component is non-symmetrical and includes a built-up lip around a portion of the hemispherical bearing surface to reduce the likelihood that an implanted spherically-shaped head may become dislocated from the hemispherical bearing surface.

Examples of known implantable prosthetic hip-joint replacements which address some of the aforementioned problems using an acetabulum cup assembly having locking mechanism appear in U.S. Pat. Nos. 5,049,158 and 4,380,090. In particular, the latter patent discloses that a retaining-ring for an acetabulum cup assembly is “preferably made of a resistant metal such as vitallium or stainless steel”.

Retaining-rings made of other materials such as silicone and Ultra-High Molecular Weight Polyethylene (“UHMWPE”) are also known. However, many of these alternative materials exhibit problems. For example, acetabulum cup assemblies which include either a silicone or UHMWPE retaining-ring have exhibited a wide range of push-in and push-out force. Such acetabulum cup assemblies having a retaining-ring have also exhibited problems related to the shell/insert interface being too loose, and also restrict the ROM.

U.S. Pat. No. 4,936,855 discloses an implantable prosthetic hip-joint replacement having an acetabulum cup assembly adapted for receiving a femur ball. The acetabulum cup assembly includes an insert having a stepped entry that provides a cavity which receives the femur ball. The insert's stepped entry receives a split retaining-ring. A ball shaped portion of the prosthesis introduced into the entry displaces the locking ring inwardly into a larger stepped portion of the entry so the locking ring can expand to allow passage of the ball. After the ball passes the split retaining-ring, the ring contracts and slides over the ball to a locking position in a smaller portion of the insert's stepped entry.

U.S. Pat. No. 5,782,930 also discloses an a implantable prosthetic hip-joint replacement having an acetabulum cup assembly adapted for receiving a femur ball. The acetabulum cup assembly includes:
1. an insert bearing component that receives the femur ball;
2. an outer shell component for attachment to an acetabulum to replace a natural hip socket which includes a cavity for receiving the insert bearing component therein; and
3. a retaining-ring for interlocking the femur ball into the outer shell.
   In one embodiment, the retaining-ring simultaneously fits into grooves located in the components being interlocked. The disclosed retaining ring is preferably made entirely from a polyetheretherketone (“PEEK”) material. Other embodiments of the retaining ring include adding a reinforcing material to the PEEK such as carbon fiber.

U.S. Pat. No. 6,916,342 discloses an a constrained implantable prosthetic hip-joint replacement that includes:
1. an implantable prosthetic cup having a cavity that is adapted to receive a liner;
2. a liner having a cavity adapted to receive a generally spherically-shaped implant stem head;
3. a member connected to the liner which is adapted to impede the implant stem head from escaping the cavity; and
4. an adapter component.
   The adapter component includes:
1. a first adapter element having a first surface adapted to mate with the prosthetic cup, and a tapered second surface adapted to mate with a second adapter element; and
2. a second adapter element having a first surface that is tapered thereby adapting it to mate with the second surface of the first adapter element.
   When coupled, the mated tapered surfaces of the first and second adapter elements form a locking interface therebetween that constrains the liner against disassembly.

Accordingly, it is desirable to provide an improved retaining-ring for an acetabulum cup assembly. It is also desirable that an improved retaining-ring for an acetabulum cup assembly be mass-producible particularly to assure consistency in the locking mechanism and to assure the quality of the acetabulum cup assembly's components. Moreover, it is desirable to provide an acetabulum cup assembly having a locking mechanism that exhibits consistent push-in and pull-out forces.

### DISCLOSURE

An object of the present disclosure is to provide an improved prosthetic hip-joint having a femoral head which is constrained against dislocation.

Another object of the present disclosure is to provide an improved prosthetic hip-joint having a femoral head which is constrained against dislocation that is able to rotate through a planar angle which exceeds at least one-hundred fifty-three degrees)(153°).

Yet another object of the present disclosure is to provide an improved prosthetic hip-joint having a femoral head which is constrained against dislocation by a preestablished amount of force which is adjustable during implantation of the prosthetic hip-joint.

Yet another object of the present disclosure is to provide an improved prosthetic hip-joint which improves rehabilitation from an implantation procedure.

Yet another object of the present disclosure is to provide an improved prosthetic hip-joint which reduces the time interval required to heal from an implantation procedure.

Yet another object of the present disclosure is to provide an improved prosthetic hip-joint which by permitting a greater ROM reduces the time interval required to heal from an implantation procedure.

Briefly, an improved prosthetic hip-joint in accordance with the present disclosure includes:
a. a prosthetic acetabulum cup that is adapted for implantation into a pelvis bone;
b. a prosthetic femoral assembly which includes:
   i. a prosthetic, ball-shaped femoral head that during implantation becomes located within the prosthetic acetabulum cup; and
   ii. a prosthetic femoral stem that is fixed at a first end to the prosthetic, ball-shaped femoral head, and that has a second end distal from the first end which is adapted for implantation into a medullary canal of a femur; and
c. a prosthetic liner assembly adapted to be secured to the acetabulum cup and is also adapted to receive and to constrain the femoral head against dislocation.
   In one aspect of the present disclosure, the prosthetic hip-joint permits the femoral head to rotate through a planar angle which exceeds at least one-hundred fifty-three degrees) (153°) while concurrently constraining the femoral head against dislocation. In another aspect of the present disclosure, the prosthetic hip-joint constrains the femoral head against dislocation by a preestablished amount of force which is adjustable during implantation thereof.

These and other features, objects and advantages of the present disclosure will be understood or apparent to those of ordinary skill in the art from the following detailed description of various embodiments illustrated in the drawing figures.

### BRIEF DESCRIPTION OF DRAWINGS

FIG. 1 is a perspective diagram illustrating an extended range of motion, constrained prosthetic hip-joint in accordance with the present disclosure associated with a human pelvis bone;

FIG. 2 is an exploded perspective diagram illustrating in greater detail the prosthetic hip-joint depicted in FIG. 1;

FIG. 3 is a perspective diagram illustrating an acetabulum cup included the prosthetic hip-joint depicted in FIGS. 1 and 2;

FIG. 4 is a perspective diagram illustrating in greater detail a liner assembly, included in the prosthetic hip-joint in depicted in FIG. 1, with a securing bolt mated with and projecting outward from a rotating liner included in the liner assembly, the rotating liner having both a constraining ring and a tension adjusting circlip encircling an end thereof which is furthest from the projecting securing bolt;

FIG. 5 is a perspective diagram illustrating in greater detail the constraining ring depicted in FIGS. 1, 2 and 4;

FIG. 6 is a perspective diagram illustrating the constraining ring depicted in FIG. 5 mated with an installation tool used while assembling the circlip onto the rotating liner;

FIG. 7 is a perspective diagram illustrating in greater detail the tension adjusting circlip depicted in FIGS. 1, 2 and 4;

FIG. 8 is an exploded, cross-sectional perspective diagram illustrating a cage assembly adapted for securing the liner assembly of the prosthetic hip-joint depicted in FIGS. 1 and 2 to the acetabulum cup by rotating the securing bolt;

FIGS. 9A through 9C are perspective diagrams illustrating the tension adjusting circlip depicted in FIG. 7 mated with the constraining ring depicted in FIG. 5 in differing orientations; and

FIGS. 10A through 10C are cross-sectional elevational views of the assembled constrained prosthetic hip-joint in differing orientations which demonstrates the enhanced ROM provided by the disclosed prosthetic hip-joint.

### BEST MODE FOR CARRYING OUT THE DISCLOSURE

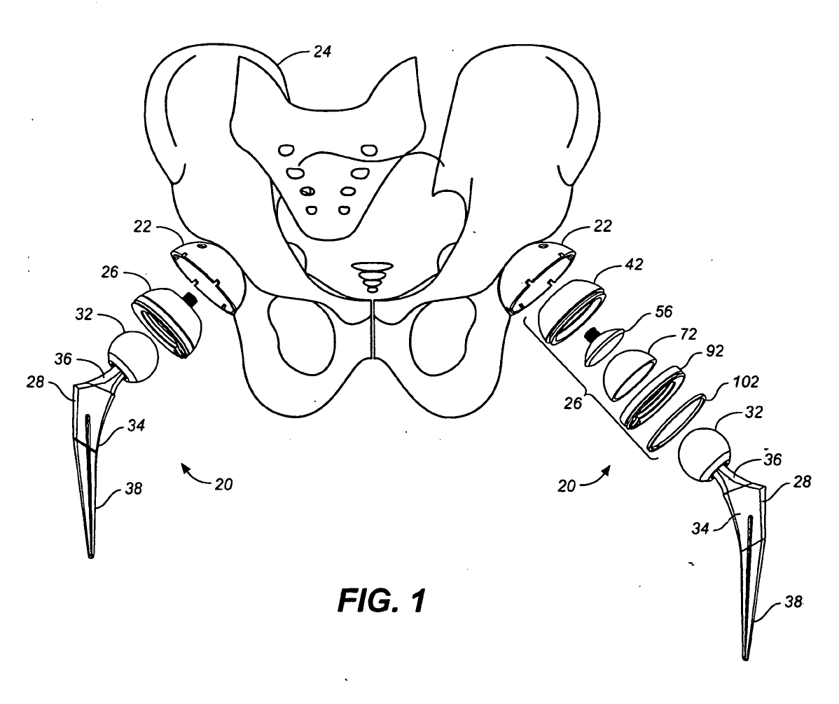

The perspective diagram of FIG. 1 depicts an identical pair of extended range of motion, constrained prosthetic hip-joints in accordance with the present disclosure referred to by the general reference character 20. The prosthetic hip-joint 20 appearing on the left hand side of FIG. 1 illustrates major subassemblies making up the prosthetic hip-joint 20 which include a prosthetic acetabulum cup 22 that is adapted for implantation into a pelvis bone 24. The prosthetic hip-joint 20 also includes a liner assembly 26, described in greater detail below, which during implantation of the prosthetic hip-joint 20 is fixed to the acetabulum cup 22.

The prosthetic hip-joint 20 further includes a prosthetic femoral assembly 28. The femoral assembly 28 includes a prosthetic, ball-shaped femoral head 32 and a prosthetic femoral stem 34. A first end 36 of the femoral stem 34 is fixed to the femoral head 32 while a second end 38 of the femoral stem 34 distal from the first end 36 is adapted for implantation into a medullary canal of a femur. Except for possible material selections described in greater detail below, the femoral head 32 and the femoral stem 34 are conventional. Securing the liner assembly 26 into the acetabulum cup 22 permits the femoral head 32 to be received into the liner assembly 26 within the acetabulum cup 22 as described below.

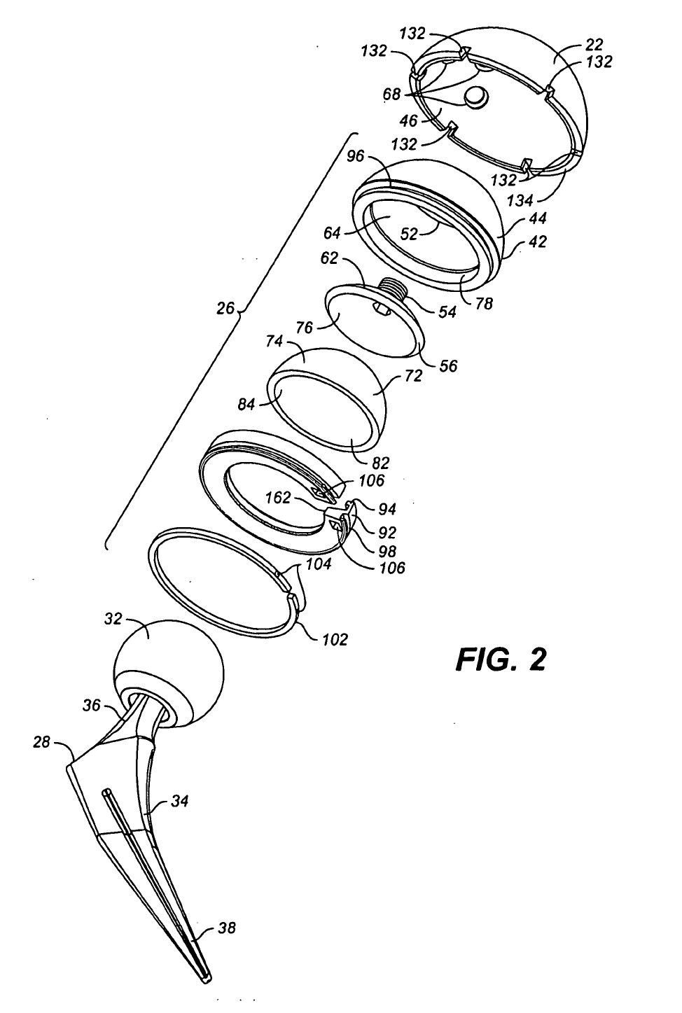

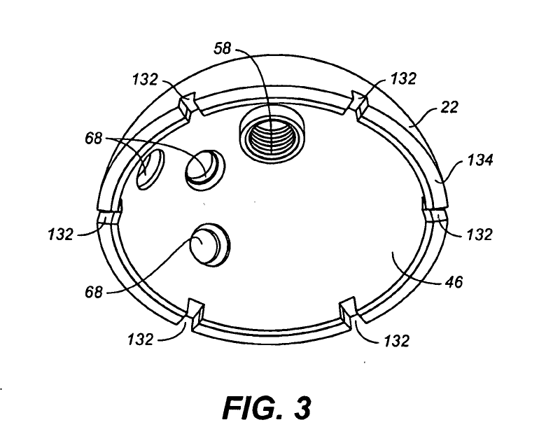

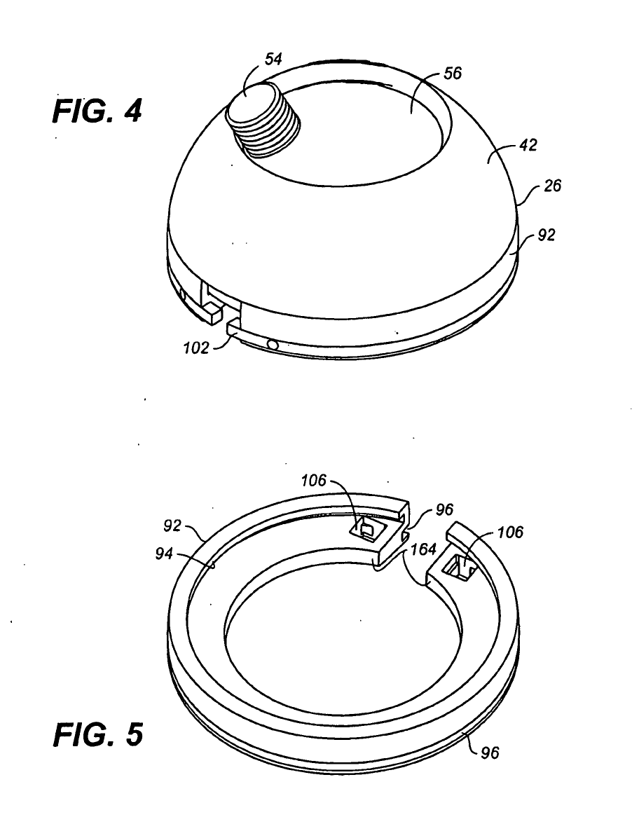

The exploded perspective illustration of the prosthetic hip-joint 20 appearing on the right hand side of FIG. 1 as well as the exploded perspective diagram of FIG. 2 illustrate in greater detail the liner assembly 26 depicted at the right hand side of FIG. 1. As illustrated in FIGS. 1 and 2, the liner assembly 26 includes a rotating liner 42 having a spherically-shaped outer surface 44 which mates with a spherically-shaped inner surface 46 of the acetabulum cup 22. An aperture 52 passing through the rotating liner 42 accommodates a threaded stem 54 of a securing bolt 56 also included in the liner assembly 26. The stem 54 of the securing bolt 56 screws into and mates with an inwardly projecting boss 58 of the acetabulum cup 22 best illustrated in FIGS. 3 and 4. The stem 54 of the securing bolt 56 projects outward from a relatively large, spherically-shaped head 62 of the securing bolt 56 which mates with a spherically-shaped inner surface 64 of the rotating liner 42. As is readily apparent from the illustration of FIG. 4, the aperture 52 piercing the rotating liner 42 has a much larger diameter than that of the stem 54 of the securing bolt 56, and the head 62 of the securing bolt 56 is larger in diameter than the aperture 52. Thus, when the securing bolt 56 secures the rotating liner 42 to the acetabulum cup 22 the rotating liner 42 remains free to move within the acetabulum cup 22 with respect to the securing bolt 56.

As also depicted in FIGS. 3 and 4, the acetabulum cup 22 is pierced by several cancellous bone screw apertures 68. The screw apertures 68 receive conventional cancellous bone screws, not illustrated in any of the FIGs., for securing the acetabulum cup 22 to the pelvis bone 24 during implantation of the prosthetic hip-joint 20.

As also illustrated in FIGS. 1 and 2, the liner assembly 26 further includes a spherically-shaped femoral ball liner 72 having an outer surface 74 which mates with a spherically-shaped surface 76 formed on the head 62 of the securing bolt 56. The open inner surface 64 of the rotating liner 42 furthest from the aperture 52 includes an inwardly projecting circularly-shaped rim 78 which surrounds the perimeter of the head 62 of the securing bolt 56, and which mates with and supports the outer surface 74 of the femoral ball liner 72 about an open end 82 thereof. A spherically-shaped inner surface 84 of the femoral ball liner 72 is shaped to receive and mate with the femoral head 32 of the femoral assembly 28.

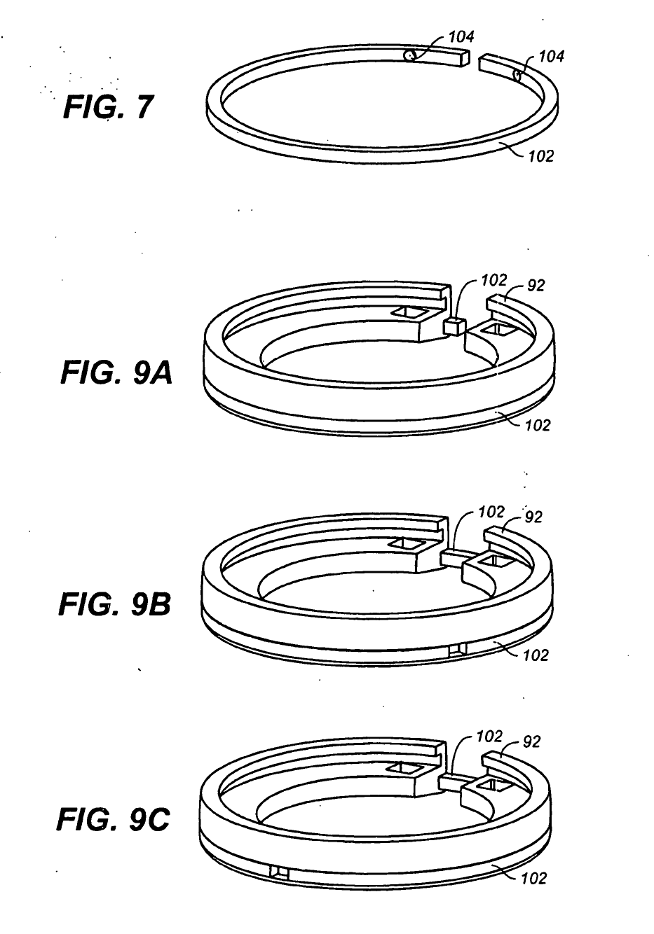

The liner assembly 26 also includes a split constraining ring 92, best illustrated in FIG. 5. The constraining ring 92 includes an inward facing lip 94 that is shaped to be received into and mated with a groove 96 that encircles the rotating liner 42 about the outer surface thereof. The constraining ring 92 in turn includes a groove 98 that encircles the outer surface thereof that is furthest from the rotating liner 42 when the lip 94 is received into and mated with the groove 96. Finally, the liner assembly 26 includes a split, tension adjusting circlip 102, best illustrated in FIG. 7, that is received into and mated with the groove 98 of the constraining ring 92.

The liner assembly 26 depicted in FIG. 4 is preferably pre-assembled while fabricating the prosthetic hip-joint 20 by first inserting the stem 54 of the securing bolt 56 through the aperture 52 of the rotating liner 42 so the head 62 of the securing bolt 56 mates with the inner surface 64 of the rotating liner 42. Then the femoral ball liner 72 is inserted into the rotating liner 42 to contact and mate with both the surface 76 of the securing bolt 56 and the inwardly projecting rim 78 of the rotating liner 42. With the securing bolt 56 and the femoral ball liner 72 located in the rotating liner 42, the lip 94 of the constraining ring 92 is then mated with the groove 96 of the rotating liner 42. Two (2) slots 106, depicted in FIG. 5, that respectively pierce the split constraining ring 92 adjacent to opposite ends thereof facilitate expanding the constraining ring 92 for installation into the groove 96 of the rotating liner 42.

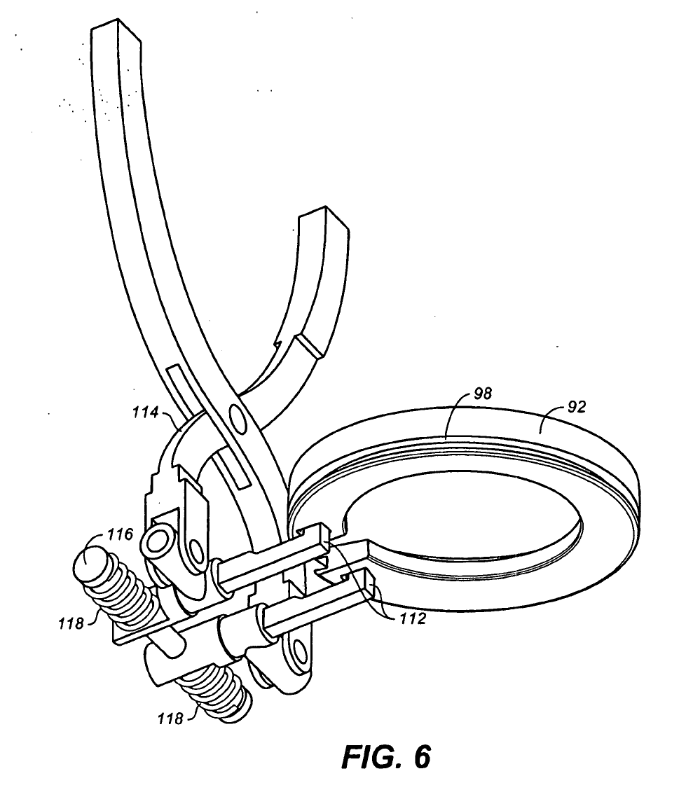

The slots 106 of the constraining ring 92 are respectively adapted to mate with tangs 112 of a pliers-like constraining ring installation tool 114 depicted in FIG. 6. Tips of each of the tangs 112 respectively include a hook, not illustrated in any of the FIGs. adapted to be received into mating recesses respectively formed in walls of the slots 106 that are nearest to confronting ends of the constraining ring 92. Thus, when the tangs 112 are positioned in the slots 106 the hooks confront each other across the split in the constraining ring 92. A minimum spacing between the tangs 112 is adjusted by means of a threaded bolt 116 that extends through springs 118 that are respectively located opposite sides of the tangs 112. When the lip 94 of the constraining ring 92 is fully seated in the groove 96 of the rotating liner 42, the tangs 112 contact opposite surfaces of the slots 106 furthest from the split in the constraining ring 92 and can be easily removed therefrom because the hooks of the tangs 112 disengage from the recesses in the walls of the slots 106. If the lip 94 of the constraining ring 92 does fully seat in the groove 96 of the rotating liner 42, the hooks of the tangs 112 remain engaged with the recesses in the walls of the slots 106 thereby preventing removal of the tangs 112 from the slots 106. An inability to remove the tangs 112 from the slots 106 probably indicates that debris is present between the constraining ring 92 and the rotating liner 42.

After the constraining ring 92 is properly installed about the inner surface 64 of the rotating liner 42, fabrication the liner assembly 26 is completed by installing the circlip 102 into the groove 98 of the constraining ring 92. When the circlip 102 is initially installed into the groove 98, the splits in the constraining ring 92 and the circlip 102 are aligned. Functionally, the circlip 102 is basically a standard seger ring, also called a snap ring. Accordingly, two apertures 104 pierce the circlip 102 to permit its expansion using a conventional snap ring pliers during its installation into the groove 98.

Implantation of the prosthetic hip-joint 20 begins conventionally with securing the acetabulum cup 22 to the pelvis bone 24 with cancellous bone screws that pass through the screw apertures 68. It is imperative that the screw heads lay entirely within the acetabulum cup 22 thereby avoiding contact the liner assembly 26. Then the liner assembly 26 is initially secured to acetabulum cup 22 by manually threading the stem 54 of the securing bolt 56 into the projecting boss 58.

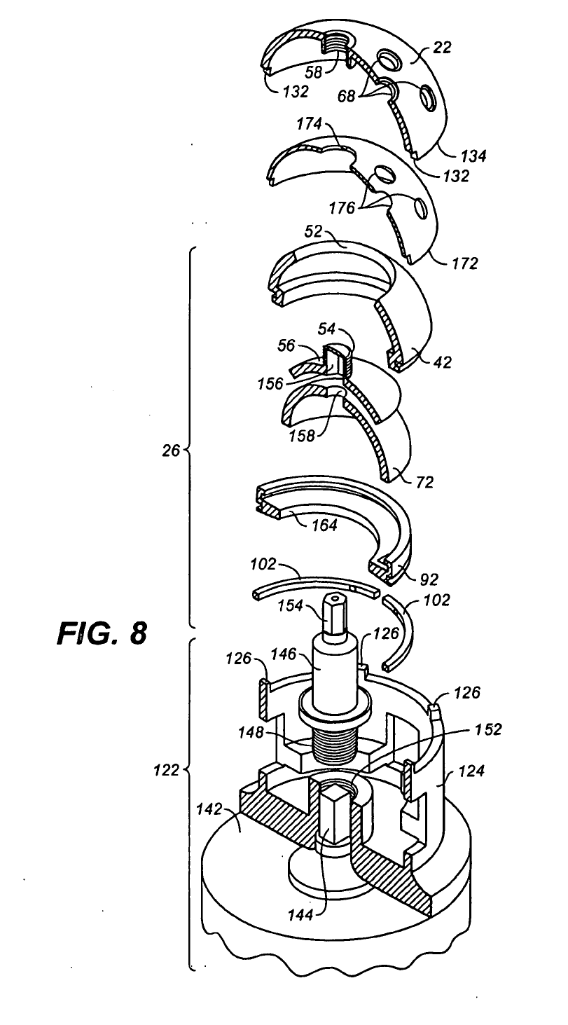

Completely fixing the liner assembly 26 to the acetabulum cup 22 is preferably performed using a cage assembly 122 depicted in FIG. 8. The cage assembly 122 includes a cylindrically-shaped cage 124 having six (6) outwardly-projecting prongs 126. While securing the liner assembly 26 to the acetabulum cup 22, the prongs 126 mate with six (6) notches 132 formed into an open, circular perimeter 134 of the acetabulum cup 22. For reasons explained in greater detail below, it is absolutely imperative that the projecting prongs 126 of the cage 124 and the notches 132 of the acetabulum cup 22 line up properly and interlock.

The cage assembly 122 also includes an electric motor 142 that is secured to the cage 124 for rotating a square drive shaft 144 very slowly with very high torque. A release clutch, not shown in any of the drawings that couples the electric motor 142 to the drive shaft 144, disconnects the electric motor 142 from the drive shaft 144 preferably when the torque on the drive shaft 144 reaches a preestablished value of 400 inch-lbs. Alternatively, the torque at which the clutch disconnects the electric motor 142 from the drive shaft 144 may be adjustable. Before mating the projecting prongs 126 with the notches 132, a pin 146, which is threaded at a first end 148 and which includes a square cavity not illustrated in any of the FIGs. within the first end 148, is fitted onto the drive shaft 144 and screwed into mating threads 152 included in the cage 124. A hexagonally-shaped Allen key 154 projects from a second end of the pin 146 furthest from the first end 148 thereof. As illustrated in FIG. 8, the stem 54 of the securing bolt 56 includes a hexagonally-shaped cavity 156 adapted to mate with the Allen key 154. Also, the wall of the femoral ball liner 72 is pierced by an aperture 158 that permits the Allen key 154 to pass therethrough while mated with the cavity 156. Furthermore, the pitch of the threads 152 of the cage 124 and the mating first end 148 of the pin 146 is identical to those on the stem 54 of the securing bolt 56 and the mating boss 58 of the acetabulum cup 22. Configured in this way, when the pin 146 is mated both to the drive shaft 144 and to the cavity 156 of the securing bolt 56 and the pin 146 rotated by the drive shaft 144, both the pin 146 and the securing bolt 56 advance synchronously toward the boss 58 of the acetabulum cup 22.

Using the motorized cage assembly 122 for tightening the securing bolt 56 with the prongs 126 of the cage 124 engaging and fully mated with the notches 132 of the acetabulum cup 22 confines all tightening torque to the mated cage assembly 122 and acetabulum cup 22. In this way the acetabulum cup 22 and the cage assembly 122 avoid transmitting any torque to the cancellous bone screws securing the acetabulum cup 22 to the pelvis bone 24 while fixing the liner assembly 26 to the acetabulum cup 22.

After the liner assembly 26 has been fastened to the acetabulum cup 22, the femoral head 32 must be installed into the femoral ball liner 72. The constraining ring 92 includes a slightly tapered inner surface 164 that is substantially coplanar with the groove 96, and is located beyond an equator of the femoral head 32 when the femoral head 32 is mated with the inner surface 84 of the femoral ball liner 72. Arranged in this way, more than one-half of the ball-shaped femoral head 32 lies between the inner surface 164 and the surface 76 of the securing bolt 56 that abuts the outer surface 74 of the femoral ball liner 72. For a femoral head 32 having a diameter of approximately one inch and four-hundred and nineteen thousandths of an inch (1.419 inch diameter), when the constraining ring 92 is properly fitted to the rotating liner 42 the inner surface 164 preferably has a diameter that is approximately seven-thousandths (0.007) of an inch smaller than the diameter of the femoral head 32. During installation of the femoral head 32 into the femoral ball liner 72, the installation tool 114 is used for expanding the constraining ring 92 approximately twenty thousandths (0.020) of an inch to allow the femoral head 32 to pass easily through the inner surface 164 and mate with the inner surface 84 of the femoral ball liner 72. After the femoral head 32 is installed within the femoral ball liner 72, wiggling the constraining ring 92 from side-to-side with respect to the rotating liner 42 with the installation tool 114 may be required to effect disengagement of the prongs 126 of the installation tool 114 from the slots 106 of the constraining ring 92.

When the ball-shaped femoral head 32 is positioned in the femoral ball liner 72 and the constraining ring 92 is fully and properly installed on the rotating liner 42, during normal hip movement with the femoral head 32 fully seated in the femoral ball liner 72 there exists a gap of approximately two and one-half thousandths (0.0025) of an inch between the inner surface 164 and the femoral head 32. If a force were applied to the femoral head 32 tending to dislocate it from the femoral ball liner 72, due to the narrow space between the inner surface 164 and the femoral head 32 initially a piston-like effect resists outward movement of the femoral head 32. The femoral head 32 would have to move outward away from the securing bolt 56 approximately fifty thousandths (0.050) of an inch against this resistance before the femoral head 32 contacts the inner surface 164 of the constraining ring 92. Dislocating the femoral head 32 from the liner assembly 26 requires applying a force to the constraining ring 92 which expands the diameter of the inner surface 164 approximately seven-thousandths (0.007) of an inch. If the femoral head 32 were to contact the inner surface 164, it is readily apparent that the combined precise shape of the ring 92 and the properties of material from which it is made constrain the femoral head 32 to remain within the liner assembly 26 by the inherent resistance of the constraining ring 92 to expanding the diameter of the inner surface 164. Furthermore, the circlip 102 located in the groove 96 of the constraining ring 92 further increases resistance to expanding the diameter of the inner surface 164 by some amount of force regardless of the orientation of the split in the circlip 102 with respect to the split in the constraining ring 92.

As described above, the constraining ring 92 in combination with the circlip 102 establish an amount of force required to dislocate the femoral head 32 from the liner assembly 26. During or even before implantation of the prosthetic hip-joint 20, differing orientations for the split in the constraining ring 92 with respect to the split in the circlip 102 permit. adjusting the preestablished amount of force required for dislocating the femoral head 32. FIGS. 9A through 9C illustrate various different orientations for the split in the circlip 102 with respect to the split in the constraining ring 92. Aligning the split in the circlip 102 with that in the constraining ring 92 as depicted in FIG. 9A offers the lowest resistance to dislocation determined essentially by the shapes and material properties both of the constraining ring 92 and of the circlip 102. Displacing the splits in the circlip 102 with respect to that in the constraining ring 92 by approximately one-hundred thirty-five degrees)(135°) from each other as depicted in FIG. 9B increases the force required to expand the diameter of the inner surface 164 by approximately 30% to 40% from that for alignment of the splits depicted in FIG. 9A. When, as depicted in FIG. 9C, the splits in the circlip 102 with respect to the split in the constraining ring 92 are diametrically opposed, i.e. oriented one-hundred eighty degrees) (180°) from each other, the constraining ring 92 becomes virtually solid, i.e. it behaves as though it lacks the split. This characteristic for the constraining ring 92 is due to the fact that a force tending to expand the diameter of the inner surface 164 must exceed the yield strength of the circlip 102. Consequently, the orientations depicted in FIG. 9C makes it virtually impossible to dislocate the femoral head 32 from the liner assembly 26. Accordingly, this latter orientation for the constraining ring 92 and the circlip 102 risks dislocating the acetabulum cup 22 and the cancellous screws passing therethrough from the pelvis bone 24.

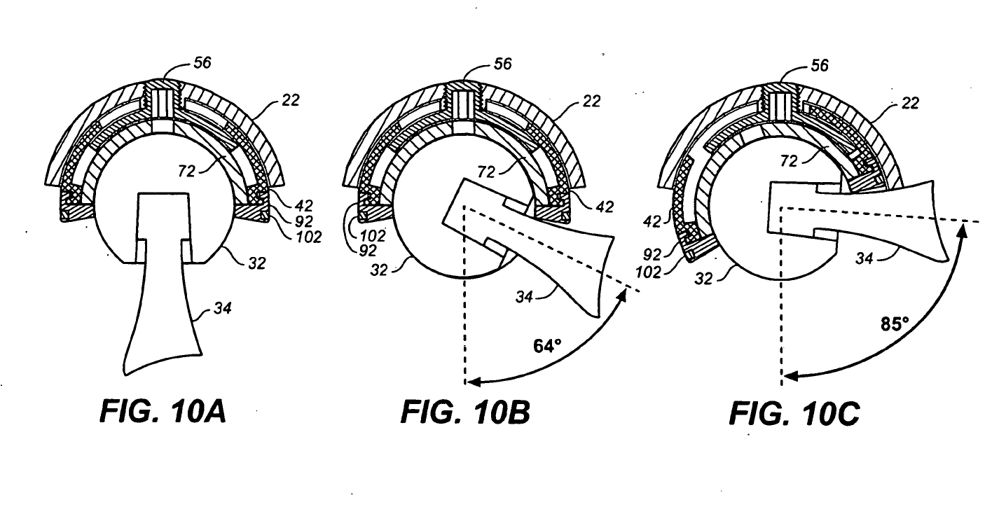

The cross-sectional views of the prosthetic hip-joint 20 appearing in FIGS. 10A through 10C depict various orientations for the femoral head 32 with respect to the acetabulum cup 22 to graphically display the enhanced ROM provided by the prosthetic hip-joint 20. FIG. 10A depicts the femoral head 32 in a central orientation with respect to the acetabulum cup 22, i.e. unrotated. FIG. 10B depicts the femoral head 32 after rotating sixty-four degrees)(64°) counterclockwise from the orientation depicted in FIG. 10A. During such a sixty-four degrees)(64°) rotation of the femoral head 32 the rotating liner 42 need not move with respect to the acetabulum cup 22 and securing bolt 56. As is readily apparent from FIG. 10C, movement of the rotating liner 42 with respect to the acetabulum cup 22 when the femoral stem 34 contacts the constraining ring 92 permits orientating the femoral head 32 up to eighty-five degrees)(85°) from that depicted in FIG. 10A. The eighty-five degrees)(85°) orientation for the rotating liner 42 with respect to the acetabulum cup 22 depicted in FIG. 10C occurs while the femoral head 32 remains constrained within the liner assembly 26 by the circlip 102 and the constraining ring 92.

Because the prosthetic hip-joint 20 permits the constrained femoral head 32 to rotate eighty-five degrees)(85°) both clockwise and counterclockwise in the same plane with respect to the acetabulum cup 22 from the unrotated orientation depicted in FIG. 10A, the prosthetic hip-joint 20 permits a total ROM for the femoral head 32 of one-hundred and seventy degrees)(170°) rotation in any arbitrarily chosen plane passing through the center of the femoral head 32. Clearly, a one-hundred and seventy degrees)(170°) ROM in any arbitrarily chosen plane passing through the center of the femoral head 32 provides the prosthetic hip-joint 20 with a ROM that significantly exceeds a one-hundred and fifty-three)(153°) rotation in any such plane. Furthermore, the one-hundred and seventy degrees)(170°) ROM occurs while the femoral head 32 is simultaneously constrained against dislocation from the acetabulum cup 22 by a preestablished amount of force which is adjustable during implantation of the prosthetic hip-joint 20.

### Industrial Applicability

As is readily apparent to those skilled in the art, everything included in the prosthetic hip-joint 20 either must be made entirely from a biocompatible material, or may have an internally located non-biocompatible material that is entirely encased within an impermeable layer of a biocompatible material.

It is also readily apparent that different size prosthetic hip-joints 20 are required to provide a proper fit to a particular individual. In the following descriptions, various different component dimensions of the prosthetic hip-joint 20 are intended for use with a femoral head 32 having a diameter of approximately one inch and four-hundred and nineteen thousandths of an inch (1.419 inch diameter).

Acetabulum Cup 22

While the acetabulum cup 22 may be made of a biocompatible cobalt-chrome material, for various reasons alternative biocompatible materials may be preferable. For example, the acetabulum cup 22 could be made from titanium such as titanium 6AL4V, or from carbon-carbon material. However, making the acetabulum cup 22 from titanium requires interposing an intermediate liner 172 depicted in FIG. 8 between the acetabulum cup 22 and the rotating liner 42 to compensate for titanium's unacceptable bearing characteristics. If the material selected for the acetabulum cup 22 requires including the intermediate liner 172 in the prosthetic hip-joint 20, the intermediate liner 172 must:
1. include an aperture 174 which fits around the boss 58;
2. include apertures 176 which mate with the cancellous bone screw apertures 68 that pierce the acetabulum cup 22; and
3. be fixed to the pelvis bone 24 by heads of the cancellous bone screws.
   The intermediate liner 172 may be made from:
1. a carbon fiber re-enforced PEEK material perhaps such as PEEK CR manufactured by Invibio Ltd. of Technology Centre, Hillhouse International Thornton Cleveleys, Lancashire, United Kingdom (“Invibio”); or
2. pure PEEK Optima also manufactured by Invibio.

Whichever material is chosen for the acetabulum cup 22, the outer surface juxtaposed with the pelvis bone 24 should have a coating of porous titanium. This porous titanium coating can be applied by metal spraying, plasma spraying or vapor deposition. Whichever material is selected for the acetabulum cup 22 and possibly for the intermediate liner 172, the inner surface thereof juxtaposed with the outer surface 44 of the rotating liner 42 must be highly polished to permit smooth movement of the rotating liner 42.

For a femoral head 32 having a diameter of approximately one inch and four-hundred and nineteen thousandths of an inch (1.419 inch diameter), the outer surface of the acetabulum cup 22 is preferably formed with a radius of approximately one inch and two-hundred and twenty thousandths of an inch (1.220 inches). If due to the particular material selected for the acetabulum cup 22 the prosthetic hip-joint 20 lacks a intermediate liner 172, such a acetabulum cup 22 has a wall thickness of approximately one-hundred and fifty thousandths of an inch (0.150 inch). If the prosthetic hip-joint 20 includes the intermediate liner 172, the acetabulum cup 22 has a wall thickness of approximately one-tenth of an inch (0.100 inch), and the intermediate liner 172 has a wall thickness of fifty thousandths of an inch (0.050 inch).

Rotating Liner 42

The rotating liner 42 may be made from any one of four (4) different materials or combination of materials listed below.

Materials which may be used for the rotating liner 42 include:
1. a cobalt-chrome material;
2. a carbon fiber reinforced PEEK material;
3. carbon-carbon; or
4. PEEK or polyethylene reinforced with carbon-carbon.
   If the rotating liner 42 is made from a polymeric material such as PEEK or polyethylene, because during a ROM exceeding one-hundred twenty-eight degrees)(128°) the rotating liner 42 moves with respect to the securing bolt 56 while concurrently supporting the constraining ring 92, the polymeric material must be reinforced, e.g. by carbon fiber or carbon-carbon. Carbon-carbon reinforcing may possibly be required if the material forming the rotating liner 42 is cross-linked polyethylene. Carbon-carbon proves to be the strongest and toughest of all the materials identified above. A paper entitled “Biological Response to Wear Debris Generated in Carbon Based Composites as Potential Bearing Surfaces for Artificial Hip Joints,” Howling, et al., Journal of Biomedical Materials Research Part B: Applied Biomaterials, Volume 67B, Issue 2, Pages 758-764, 2003, reports that carbon-carbon material exhibits acceptable debris deposition and durability characteristics. The thickness and finish of the rotating liner 42 is of utmost importance. Proper movement of the rotating liner 42 depends on the accuracy of its manufacture.

Making the rotating liner 42 or other part of the prosthetic hip-joint 20 from a polymeric material such as PEEK reinforced with carbon-carbon, the carbon-carbon reinforcement must be completely embedded within the biocompatible polymeric material. For such reinforcements the carbon-carbon must be woven and processed to yield a very porous mesh before being mated with the polymeric material. Such an extremely strong, porous carbon-carbon reinforcement is then placed in an injection mold and the polymeric material injected concurrently on both sides of the mold thus keeping the carbon-carbon reinforcement centered in the finished product such as the rotating liner 42.

For a femoral head 32 having a diameter of approximately one inch and four-hundred and nineteen thousandths of an inch (1.419 inch diameter), the outer surface of the rotating liner 42 is preferably formed with a radius of approximately one inch and sixty-seven thousandths of an inch (1.067 inch). Such a acetabulum cup 22 has a wall thickness of approximately one-hundred twenty thousandths of an inch (0.120 inch). The aperture 52 has a diameter at the outer surface of the rotating liner 42 of approximately one inch and three-hundred twenty-five thousandths of an inch (1.325 inch) and at the inner surface of approximately one inch and two-hundred twenty thousandths of an inch (1.220 inch).

Securing Bolt 56

The securing bolt 56 must be made of cobalt-chrome or the highest quality stainless steel material. The surface 76 of the securing bolt 56 that is juxtaposed with the outer surface 74 of the femoral ball liner 72 must be accurate and highly polished. The external thread on the stem 54 of the securing bolt 56 is preferably 0.375 inches in diameter, 0.375 inches long and has a pitch of 24 threads per inch (“TPI”). The thread on the stem 54 must be class 3A, which has a tolerance of 0.0000 inch. The mating internal thread within the boss 58 of the acetabulum cup 22 must be class B that has a tolerance of 0.0000 inch. The mating threads of the stem 54 and the boss 58 respectively being class 3A and B and being tightened to a torque of 400 inch-lbs as described above ensures that the securing bolt 56 will not loosen.

For a femoral head 32 having a diameter of approximately one inch and four-hundred and nineteen thousandths of an inch (1.419 inch diameter), the head 62 of the securing bolt 56 is preferably formed with an outer surface having a radius of approximately nine-hundred forty-four thousandths of an inch (0.944 inch). The head 62 preferably has a wall thickness of approximately one-hundred ten thousandths of an inch (0.110 inch), and a radius of curvature for the inner surface of the securing bolt 56 of approximately eight-hundred thirty-five thousandths of an inch (0.835 inch). The head 62 preferably subtends a half-angle of fifty degrees)(50°) measured at its center of curvature.

Femoral Ball Liner 72

The femoral ball liner 72 may be made from:
1. a carbon fiber reinforced PEEK material;
2. pure PEEK-Optima; or
3. carbon-carbon.
   Carbon-carbon has been demonstrated to be the most suitable for extended wear and biocompatibility.

For a femoral head 32 having a diameter of approximately one inch and four-hundred and nineteen thousandths of an inch (1.419 inch diameter), the outer surface of the femoral ball liner 72 is preferably formed with a radius of approximately eight-hundred thirty-three thousandths of an inch (0.833 inch), and a wall thickness of approximately one-hundred and twenty-two thousandths of an inch (0.122 inch).

Constraining Ring 92

Because the constraining ring 92 is the major component for resisting unintended dislocation of the femoral head 32 from the liner assembly 26, it must be made of a high quality metal and heat-treated to have characteristics equivalent to spring steel. The constraining ring 92 can be cobalt-chrome, spring steel grade stainless steel or titanium which must also be spring steel grade. This part has to be made to the highest standards of accuracy and the surface finish has to he exceptional. The split in the constraining ring 92 must be sufficiently large to ensure that the constraining ring 92 attaches firmly to the rotating liner 42. From a mechanical engineering perspective, firm attachment between the constraining ring 92 and the rotating liner 42 indicates that the split between confronting ends of the constraining ring 92 should not be smaller than fifteen thousandths (0.015) of an inch. However, determining the size of the split in the constraining ring 92 requires considering Wolff's law which concerns the ingress of human tissue towards foreign objects present in the human body.

For a femoral head 32 having a diameter of approximately one inch and four-hundred and nineteen thousandths of an inch (1.419 inch diameter), the constraining ring 92 is preferably formed with a radius for the outer circular surface of approximately one inch and forty-six thousandths of an inch (1.046 inch). The inner surface 164 of the constraining ring 92 is preferably formed with a radius of approximately seven-hundred ten thousandths of an inch (0.710 inch), and subtends an angle of approximately ten degrees)(10°) measured at its center of curvature which lies in the plane of the constraining ring 92 that abuts the open end of the rotating liner 42.

Circlip 102

Similar to the constraining ring 92, the circlip 102 can be cobalt-chrome, spring steel grade stainless steel or titanium which must also be spring steel grade. The finish of the circlip 102 is preferably left rough on the inner circular surface while the outer circular surface must be smooth and polished to ensure that human tissue does not easily migrate towards it and bond. To increase the circlip's resistance to expansion by the constraining ring 92, it may be advantageous to transversely knurl the inner circular surface of the circlip 102 which contacts the groove 96 of the constraining ring 92. From a mechanical engineering perspective, the split in the constraining ring 92 is preferably one-eighth (0.125) of an inch. However, this gap could be smaller, possibly as small as fifteen thousandths (0.015) of an inch. Similar to the constraining ring 92, determining the size of the split in the circlip 102 requires considering Wolff's law which concerns the ingress of human tissue towards foreign objects present in the human body.

For a femoral head 32 having a diameter of approximately one inch and four-hundred and nineteen thousandths of an inch (1.419 inch diameter), the circlip 102 is preferably formed with an outer surface having a radius of approximately one inch and twenty-eight thousandths of an inch (1.028 inch), and an inner surface having a radius of approximately nine-hundred and forty-eight thousandths of an inch (0.948 inch). The circlip 102 is preferably formed with a thickness of approximately seventy-seven thousandths of an inch (0.077 inch).

Femoral Head 32

As stated previously, the femoral head 32 included in the prosthetic hip-joint 20 can be entirely conventional. Consequently, giving due consideration to the service life of the prosthetic hip-joint 20 the femoral head 32 can be made of a ceramic material or of a metal such as cobalt-chrome, stainless steel, vitallium or other metal, perhaps with a ceramic coating applied thereto.

Femoral Stem 34

As stated previously, the femoral stem 34 included in the prosthetic hip-joint 20 can be entirely conventional. However, it appears that making the femoral stem 34 from fiberglass encased in carbon fiber reinforced PEEK material or pure PEEK-Optima may be advantageous. Using high tensile strength “S” fiberglass allows forming the femoral stem 34 with a directional lay up that provides a modulus of elasticity for the femoral stem 34 similar to that of human bone. Using “S” fiberglass for the femoral stem 34 is particularly advantageous because the material resists fatigue. The auto industry has found properly fabricated fiberglass springs to be advantageous because they do not break due to fatigue. A slight spring effect, which can be designed into a femoral stem 34 fabricated using “S” fiberglass, should reduce transmission of shock to the acetabular area while avoiding fatigue failure of the femoral stem 34. Furthermore, by exhibiting mechanical properties that more closely resemble that of human bone the femoral stem 34 would conform with Wolff's law more consistently than presently used metal femoral stems 34. Encasing the “S” fiberglass in PEEK or PEEK-Optima appears to offer a synergy between properties of the two materials.

Although the present invention has been described in terms of the presently preferred embodiment, it is to be understood that such disclosure is purely illustrative and is not to be interpreted as limiting. For example, while the acetabulum cup 22 is preferably secured to the pelvis bone 24 by cancellous bone screws, the acetabulum cup 22 may also be glued thereto or anchored there by spikes extending from its outer surface. Consequently, without departing from the spirit and scope of the disclosure, various alterations, modifications, and/or alternative applications of the disclosure will, no doubt, be suggested to those skilled in the art after having read the preceding disclosure. Accordingly, it is intended that the following claims be interpreted as encompassing all alterations, modifications, or alternative applications as fall within the true spirit and scope of the disclosure.

What is claimed is:

1. An improved prosthetic hip-joint comprising at least:
a. a prosthetic acetabulum cup that is adapted for implantation into a pelvis bone; and
b. a prosthetic femoral assembly which includes:
i. a prosthetic, ball-shaped femoral head that is adapted for being received within said prosthetic acetabulum cup; and
ii. a prosthetic femoral stem that is fixed at a first end to the prosthetic, ball-shaped femoral head, a second end of the prosthetic femoral stem distal from the first end thereof being adapted for implantation into a medullary canal of a femur,
said prosthetic, ball-shaped femoral head being constrained against dislocation from said prosthetic acetabulum cup while concurrently being able to rotate through a planar angle which exceeds at least one-hundred fifty-three degrees)(153°).

2. The prosthetic hip-joint of claim 1 wherein the planar angle through which said prosthetic, ball-shaped femoral head is rotatable exceeds one-hundred fifty-five degrees) (155°).

3. The prosthetic hip-joint of claim 1 wherein the planar angle through which said prosthetic, ball-shaped femoral head is rotatable exceeds one-hundred sixty degrees)(160°).

4. The prosthetic hip-joint of claim 1 wherein the planar angle through which said prosthetic, ball-shaped femoral head is rotatable exceeds one-hundred sixty-five degrees) (165°).

5. The prosthetic hip-joint of claim 1 wherein the planar angle through which said prosthetic, ball-shaped femoral head is rotatable exceeds one-hundred sixty-nine degrees) (169°).

6. The prosthetic hip-joint of any one of claims 1 through 6 wherein said prosthetic, ball-shaped femoral head is constrained against dislocation from said prosthetic acetabulum cup by a preestablished amount of force which is adjustable during implantation of the prosthetic hip-joint.

7. An improved prosthetic hip-joint comprising at least:
a. a prosthetic acetabulum cup that is adapted for implantation into a pelvis bone; and
b. a prosthetic femoral assembly which includes:
i. a prosthetic, ball-shaped femoral head that is adapted for being received within said prosthetic acetabulum cup; and
ii. a prosthetic femoral stem that is fixed at a first end to the prosthetic, ball-shaped femoral head, a second end of the prosthetic femoral stem distal from the first end thereof being adapted for implantation into a medullary canal of a femur,
said prosthetic, ball-shaped femoral head being constrained against dislocation from said prosthetic acetabulum cup by a preestablished amount of force which is adjustable during implantation of the prosthetic hip-joint.
